(19) United States
(12) Patent Application Publication
(10) Pub. No.: US 2004/0019309 A1
(43) Pub. Date: Jan. 29, 2004

(54) Ankle brace for integration with footwear

(75) Inventors: Ronald E. Nelson, Cambridge, MN (US); Curran Lee Rode, Schulenberg, TX (US)

Correspondence Address: FOLEY & LARDNER, 150 EAST GILMAN STREET, P.O. BOX 1497, MADISON, WI, 53701-1497, US

(73) Assignee: Mueller Sports Medicine, Inc.

(21) Appl. No.: 10/207,486

(22) Filed: Jul. 29, 2002

(51) Int. Cl. A61F 5/00

(52) U.S. Cl. 602/65; 602/27

(57) ABSTRACT

An ankle brace that is configured to integrate with footwear. The ankle brace includes loops located near the forward edges of the base. After the brace is secured to the foot and ankle, footwear is applied over the ankle brace, and the laces of the footwear are passed through the loops in order to secure the footwear to the ankle brace and form an integrated structure. Relief openings in the ankle brace are also provided to enhance the conformance of the ankle brace to the foot and ankle as the shape of the foot and ankle changes across the range of motion of the foot and ankle.

### FIELD OF THE INVENTION

The present invention relates generally to the field of articles worn by persons to reduce the likelihood, severity, or exacerbation of injury to the body, and more specifically to the field of braces worn on the ankle.

### BACKGROUND OF THE INVENTION

The ankle joint connects the lower leg and the foot of a person, providing a pivot point that allows the foot to rotate relative to the lower leg. Together the lower leg, ankle joint, and foot make up a complex system that must be sturdy yet flexible in order to bear a person's weight while providing freedom of movement.

The ankle joint is one of the most frequently used joints in the body, as it is required for any activity that involves walking or running. Further, it receives relatively high levels of stress as it bears dynamic loads that are multiples of the entire weight of the body. During ambulation, and especially during strenuous sports, such as football, basketball, tennis, or soccer, quick changes in direction or uneven playing surfaces can cause the ankle to move beyond its normal range of motion, resulting in an injury commonly referred to as a sprained ankle. A sprained ankle is a source of some discomfort that can make sports less enjoyable or reduce athletic performance, and it can reduce mobility and productivity during normal day-to-day activities. For these reasons, there has long been motivation to find ways to support and protect the ankle while maintaining good freedom throughout the normal range of motion, to reduce injuries without diminishing mobility.

Ankle braces have been used for many years, in a variety of specific embodiments directed to particular applications. Although there is considerable variety in the construction of ankle braces today, these ankle braces are usually worn with some kind of footwear, such as normal shoes or boots, or athletic footwear such as football or baseball cleats, running shoes, basketball shoes, or tennis shoes. Although it is common to wear ankle braces found in the prior art with some kind of footwear, the prior art does not address problems in the combination of an ankle brace with footwear, nor does the prior art maximize the benefit of the combination by forming an integrated structure made of the footwear and the ankle brace.

Some ankle braces found in the prior art include specialized footwear which is permanently attached to the brace. However, this approach has a number of disadvantages. Although an ankle brace may be configured to fit a wide range of foot sizes, footwear is generally made to fit only a specific size, so that a larger number of specific sizes must be manufactured and kept in inventory when the footwear and ankle brace are sold as a permanently attached unit. Further, this approach limits consumer choice as to the appearance and other characteristics of the footwear permanently attached to the ankle brace, compared to an approach which uses off-the-shelf footwear in combination with an ankle brace.

Other ankle braces found in the prior art are made to fit over normal footwear to form an integrated structure, but this approach also has disadvantages. For example, this approach creates bulk which reduces clearance in the area of the foot and ankle and which impedes mobility.

In some braces found in the prior art, the ankle brace is made to fit inside normal footwear, which has a number of advantages. Such an ankle brace separates the ankle brace and footwear, so that a single ankle brace can be used with a wide range of foot sizes, reducing the number of sizes which must be manufactured and kept in inventory. Such an ankle brace lets the wearer choose their footwear according to appearance or functional characteristics unrelated to the ankle brace, so that a single ankle brace design may be used with a wide range of footwear. Finally, such an ankle brace can be manufactured at a relatively low cost, since such an ankle brace does not duplicate structure provided by footwear the wearer may already own.

However, prior ankle braces made to fit inside normal footwear are merely fitted inside the footwear without attaching the ankle brace to the footwear. These ankle braces are typically first secured to the foot and ankle using eyelets and laces provided for this purpose on the ankle brace, and then the wearer puts on their normal footwear over the ankle brace and secures the normal footwear on top of the ankle brace using eyelets and laces provided on the footwear. In such an ankle brace, the ankle brace and footwear can move relative to one another, so the combination does not take full advantage of the combined structure of the footwear and the ankle brace. Such an ankle brace is not as effective at supporting and protecting the ankle as an ankle brace which forms an integrated structure with the footwear, such as an ankle brace permanently attached to specialized footwear or an ankle brace which fits outside normal footwear.

Another problem found in prior ankle braces made to fit inside normal footwear is that the support and protection provided to the ankle is not uniform across an adequate range of normal motion. The base of prior ankle braces made to fit inside normal footwear is usually constructed so that the brace conforms closely to the ankle and foot when the brace is secured to the foot and ankle with the foot and ankle in a neutral or unflexed position. However, when the foot and ankle is moved through a normal range of motion by extending the foot either up (flexion) or down (extension), the dimensions of the foot and ankle change in a complex fashion. Because of this change in dimensions of the foot and ankle, and because the base material is inelastic, although prior ankle braces made to fit inside normal footwear may conform closely to a foot and ankle in a neutral position, those ankle braces begin to lose this close conformance as the foot and ankle move toward the ends of their range of motion and away from their neutral position. When the close conformance diminishes, the support provided by the brace also diminishes. Good support can be maintained by restricting the range of motion to avoid this change in dimension of the foot and ankle, but this is often undesirable. U.S. Pat. No. 5,681,271 discloses an ankle brace that addresses this problem by providing strategically placed elastic relief openings in the otherwise inelastic base material.

For the aforementioned reasons, there is motivation to find an ankle brace made to fit inside normal footwear which forms an integrated structure with the footwear, and which provides uniform support and protection to the ankle across an adequate range of normal motion

### SUMMARY OF THE INVENTION

The present invention relates to an ankle brace made to fit inside normal footwear which forms an integrated structure with the footwear in order to maximize protection and support, and which provides good support throughout an adequate range of normal motion of the foot and ankle. An ankle brace according to the invention includes a base made of flexible material shaped to conform closely to the foot and ankle of the wearer, preferably with eyelets and laces like those found on footwear, and additional loops which are used to tie the ankle brace to the footwear. Such an ankle brace may optionally include relief openings which enhance the conformance of the ankle brace to the foot and ankle throughout the range of normal motion of the foot and ankle.

The base of an ankle brace according to the invention is first secured to the foot and ankle using the eyelets and laces on the ankle brace, in much the same way that a boot is fastened to a foot and ankle. The laces of the ankle brace are threaded through the eyelets on the ankle brace, tightened, and tied together with a knot to secure the ankle brace to the foot and ankle. The laces of the ankle brace are preferably not threaded through the loops found on the ankle brace which are used to tie the ankle brace to the footwear. Although eyelets and laces on the ankle brace are used in a preferred embodiment, other means such as straps or hook and loop material of the type which adheres when pressed together may alternatively be used to fasten the ankle brace to the foot and ankle of the wearer.

After securing the ankle brace by itself to the foot and ankle, the wearer puts on his or her normal footwear over the ankle brace. The loops on the ankle brace are then lined up with the closest eyelets on the footwear. Starting at their free end, the laces on the footwear are removed from the eyelets on the footwear down to and including the eyelets which are lined up with the loops on the ankle brace, so that the footwear is only partially laced. Depending on whether the footwear extends above the ankle, in other words, whether the footwear is “high tops” or “low tops,” there may be a greater or lesser number of eyelets through which laces are not threaded at this point.

Next, the free ends of the laces of the footwear are threaded through the loops on the ankle brace, to tie the footwear to the ankle brace. Finally, the laces of the footwear are threaded through the eyelets on the footwear (which are lined up with the loops on the ankle brace), threaded through the remaining eyelets on the footwear, and tightened and tied normally. In other words, the laces and eyelets of the footwear are used normally, as would be done if the footwear were worn alone without the ankle brace, except that the laces of the footwear pass through the special loops on the ankle brace so that the ankle brace and the footwear are secured to the foot and ankle of the wearer as an integrated structure.

In the preceding discussion of a preferred embodiment of the ankle brace according to the invention, the shoelaces of normal footwear are passed through loops attached to the ankle brace in order to attach the footwear to the ankle brace and form an integrated structure. However, this attachment may be accomplished in a variety of other ways without departing from the spirit of the invention. For example, if the footwear includes straps to secure the footwear to the foot and ankle of the wearer, instead of or in addition to shoelaces, those straps may be passed through a loop attached to the ankle brace in order to secure the ankle brace to the footwear, as an alternative to the shoelaces discussed in the preferred embodiment. Similarly, if the footwear includes hook and loop material to secure the footwear to the foot and ankle of the wearer, instead of or in addition to shoelaces, the hook and loop material on the shoe may be pressed against complementary hook and loop material attached to the ankle brace in order to secure the ankle brace to the footwear, as an alternative to the shoelaces discussed in the preferred embodiment.

In the preceding discussion of a preferred embodiment of the ankle brace according to the invention, the loops are permanently attached to the ankle brace so that the shoelaces of the footwear must be at least partially removed in order to free the ankle brace from the footwear. However, the loops on the ankle brace may be openable loops, or the loops may be releasably attached to the ankle brace, so that the ankle brace may be more easily freed from the footwear.

In a preferred embodiment of the ankle brace according to the invention, the loop on the ankle brace is attached at one end of an elongate member whose other end is fastened to the base of the ankle brace by a rivet, allowing the elongate member and the loop to rotate or swivel about the rivet. By rotating or swiveling, the wearer can line up the special loop on the ankle brace more closely with one of the eyelets on the footwear. This alignment of the special loop with one of the eyelets on the footwear could be accomplished in other ways, for example by forming the loops as slots in the base, and providing a plurality of such loops so that at least one of the loops will line up reasonably closely with an eyelet on the footwear. The loop on the ankle brace may alternatively be attached in other ways which allow lesser or greater freedom of motion of the loop. For example, the loop may be attached in such a way that the loop cannot move at all, the loop may be detachably attached so it may be moved and reattached, or the position of the special loop may be otherwise adjustable.

In a preferred embodiment of the ankle brace according to the invention, there are two loops on the ankle brace which are made of metal, formed in a shape approximating the letter “D” (“D-shaped”), with one such loop attached to the base of the ankle brace on each side of the brace above the front superior surface of the foot. The loops may be attached to the ankle brace somewhere else. There may be only a single special loop, or more than the two special loops found in the preferred embodiment. The loop may be formed of fabric, nylon webbing, plastic, metal, or any other suitable material. The loop may be formed in a shape other than D-shaped. The loop may be formed as one or more slots cut in the base of the ankle brace.

In a preferred embodiment of the ankle brace according to the invention, the material of the base of the ankle brace is constructed of two coextensive layers, where the outside layer is a flexible but inelastic material such as vinyl, and the inside layer is a flexible and elastic material. Relief openings may be cut in the inelastic outside layer at locations where bunching or excess tension tend to occur during normal flexion and extension of the ankle, without cutting relief openings in the elastic inside layer, so that the elastic inside layer spans the relief openings. Stitching may be used to secure the boundaries of the relief openings to the elastic inside layer.

Two such relief openings may be cut as V-shaped notches on the lower part of the base near the ball of the foot, one on the medial side and the other on the lateral side of the base. Two more such relief openings may be cut as curved elongate openings approximately parallel to the front edge of the base, one on the medial side and the other on the lateral side of the base. There may be a greater or lesser number of these relief openings. The relief openings may or may not be spanned by elastic. The relief openings may have different shapes.

In a preferred embodiment of the ankle brace according to the invention, the heel opening is partially covered with an elastic strip, to provide a close fit around the heel which is able to stretch during normal motion.

### BRIEF DESCRIPTION OF THE DRAWINGS

In the drawings:

FIG. 1 is a side view of an ankle brace according to the invention installed on a foot without footwear;

FIG. 2 is a front view of an ankle brace according to the invention installed on a foot without footwear;

FIG. 3 is a back view of an ankle brace according to the invention with the laces removed and the sides spread apart;

FIG. 4 is a front view of an ankle brace according to the invention with the laces removed and the sides spread apart;

FIG. 5 is a front view of an alternate embodiment of an ankle brace according to the invention with the laces removed and the sides spread apart;

FIG. 6 is a front view of a resilient stay member made of flattened springs;

FIG. 7 is a side view of a resilient stay member made of flattened springs;

FIG. 8 is a front view of an ankle brace according to the invention installed on a foot and worn with normal “high tops” footwear;

FIG. 9 is a front view of an ankle brace according to the invention installed on a foot and worn with normal “high tops” footwear with a portion of the footwear cut away to show how the laces of the footwear are trained through the special loops;

FIG. 10 is a front view of an ankle brace according to the invention showing the construction of the interior of the rear of the brace; and

FIG. 11 is a perspective view of an ankle brace according to the invention installed on a foot, with normal “low tops” footwear being applied on top of the ankle brace.

### DETAILED DESCRIPTION OF THE INVENTION

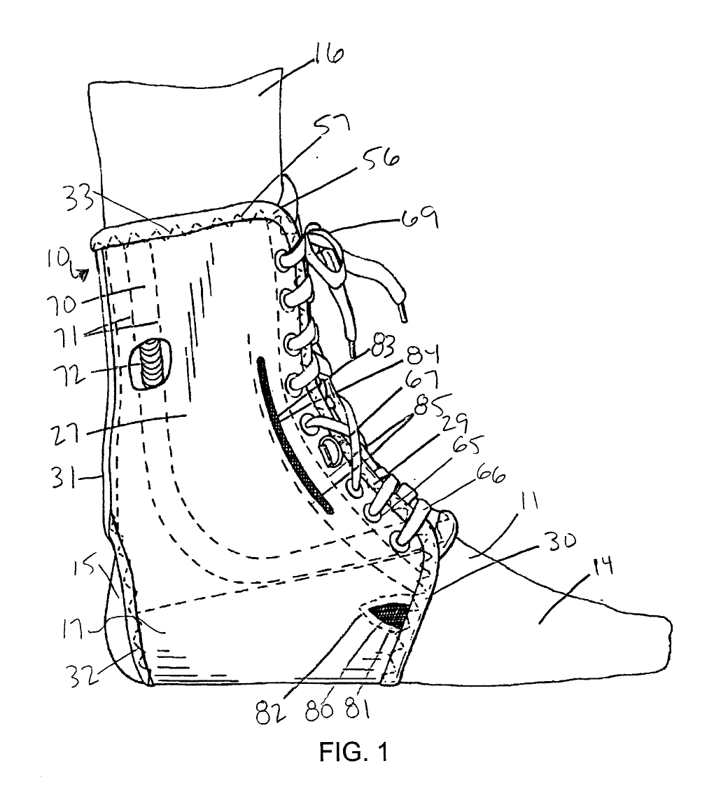

With reference to the drawings, FIG. 1 shows a preferred embodiment of an ankle brace in accordance with the invention indicated generally at 10, as worn on the right foot 11 of a person. Although FIG. 1 shows only one side of the ankle brace, the construction of the ankle brace is preferably essentially symmetric so that the side which is not shown is preferably essentially the same as the side which is shown. Although the figures and this detailed description show the ankle brace worn on the right foot, because of the symmetry of construction the ankle brace can be worn on either the left or right foot. The foot 11 has a toe region 14 that extends out from an opening in the front of the ankle brace 10. The foot also has a heel region 15 that extends out from an opening in the rear of the ankle brace. The lower leg of the person 16 extends out from an opening at the top of the ankle brace.

The base of the ankle brace 17 is preferably formed of two coextensive layers of sheet material, with the external layer 27 formed of flexible but inelastic material and the interior lining 28 (not shown in FIG. 1) formed of elastic sheet material. The base 17 has a top edge 33, a bottom edge 34 (not shown in FIG. 1), an upper forward edge 29, a lower forward edge 30, an upper rear edge 31, and a lower rear edge 32. The edges of the base 17 are covered by edge binding 56, preferably using stitching 57.

Curved elongate relief openings 83 are located near the upper forward edge 29 on each side of the base. Elastic 84 (which may be formed of the interior lining 28) spans the curved elongate relief openings 83, and is secured to the boundaries of the relief openings 83, preferably using stitching 85. V-shaped relief openings 80 are located near the lower forward edge 30 on each side of the base. Elastic 81 (which may be formed of the interior lining 28) spans the V-shaped relief openings 80, and is secured to the boundaries of the relief openings 80, preferably using stitching 82.

The ankle brace is preferably secured to the foot and ankle using a line of eyelets 65 located along the upper forward edge 29 of the base and a lace 66 which is threaded through the eyelets 65, and tied into a knot 69.

Loops 67 are located on each side of the base, approximately in line with the eyelets 65, at approximately the same vertical location as the curved elongate relief openings.

As best shown in FIG. 1, an elongate pocket 70 is preferably formed between the exterior surface 27 and the lining 28 on each side of the base, preferably by stitching 71. Although only one side is shown in FIG. 1, both sides of the ankle brace may include such an elongate pocket 70. On each side, the elongate pocket 70 preferably begins approximately where the top and upper rear edges 33 and 31 meet on that side of the base. On each side, the elongate pocket 70 then runs approximately parallel to the upper rear edge 31 on that side of the base to a point just behind and below the approximate location of the ankle, where the elongate pocket 70 changes direction to run approximately parallel to the bottom of the foot to end approximately where the upper forward edge 29 meets the lower forward edge 30 on that side of the base.

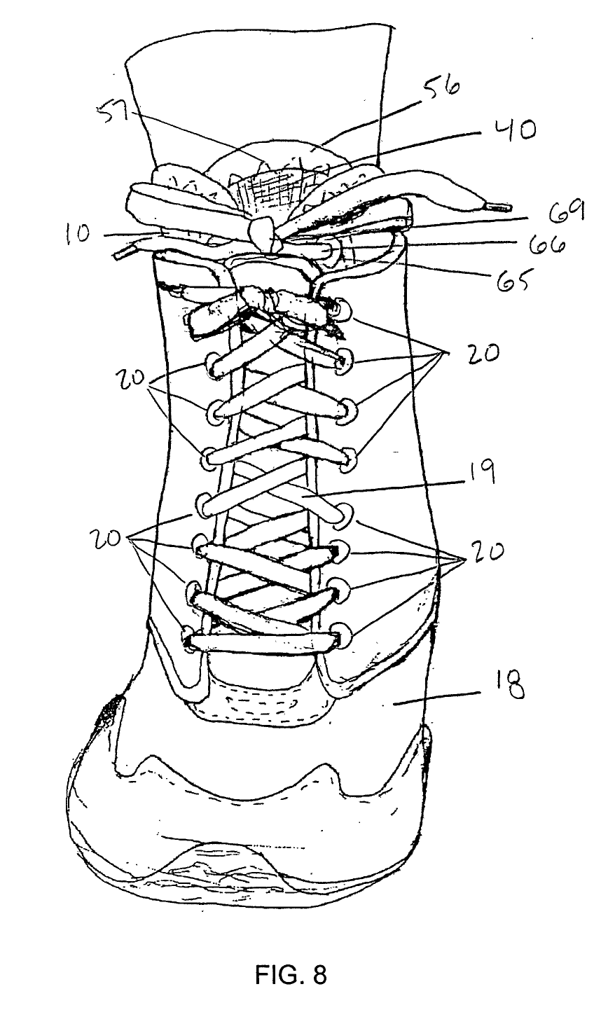

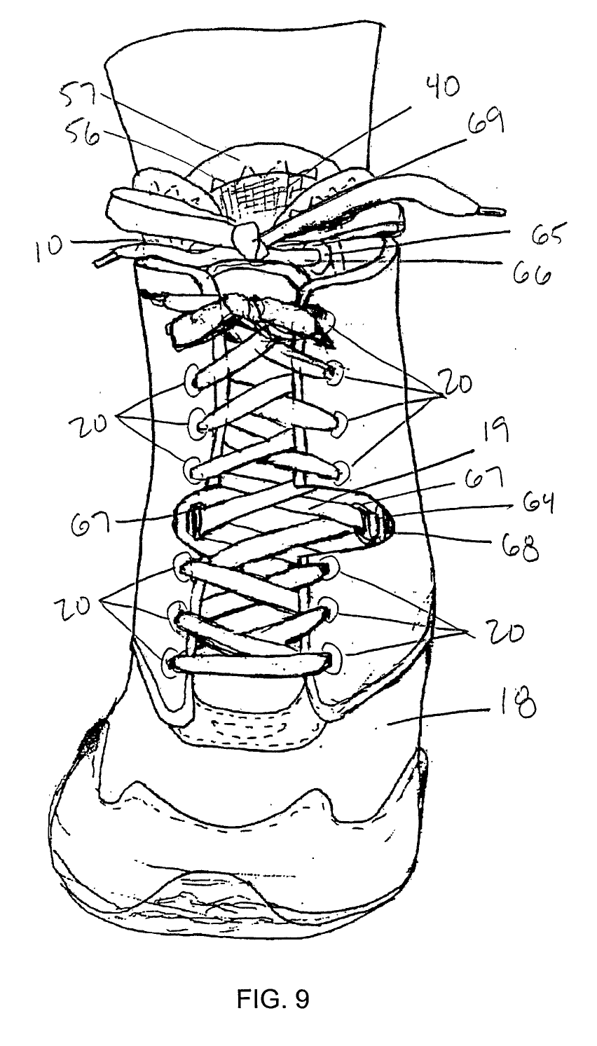

As shown in the cutaway in FIG. 1, a resilient stay member 72 is preferably located in each elongate pocket 70. As shown in FIGS. 8 and 9, the resilient stay member 72 may be formed of a pair of interleaved helical springs made of stainless steel that have been flattened. The resilient stay member 72 may alternatively be formed of other flexible material of conventional construction commonly used in various types of braces. There may be more than one elongated side pocket 70 containing a resilient stay member 72 on each side, and the elongated side pocket 70 may be openable at one end to allow removal of the resilient stay member 72 or replacement of the resilient stay member 72 with a different resilient stay member having different resiliency to adjust the amount of support provided.

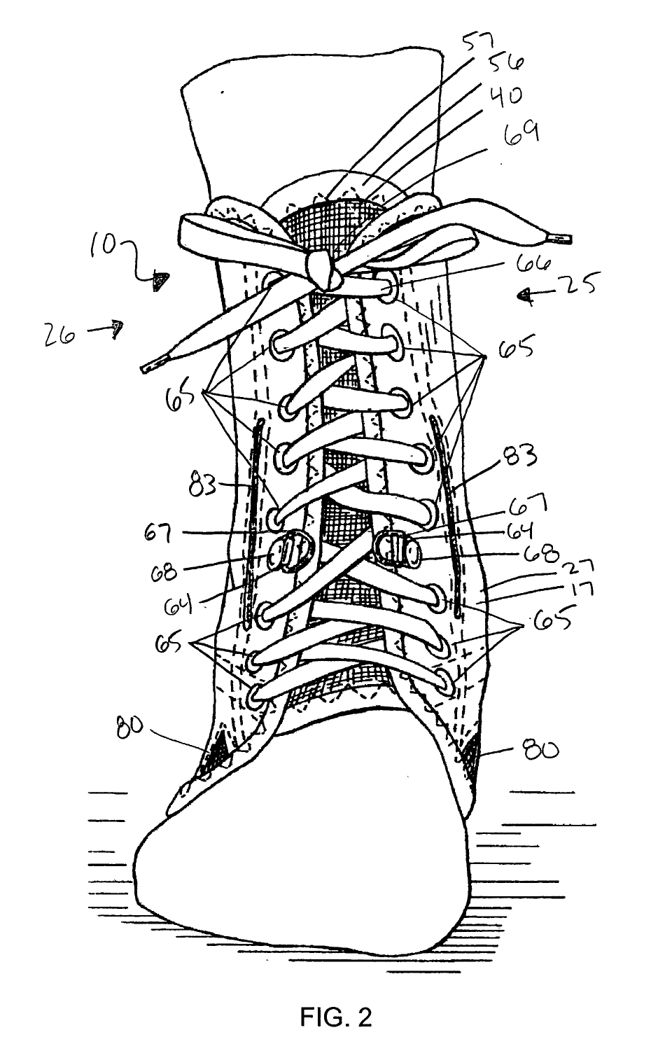

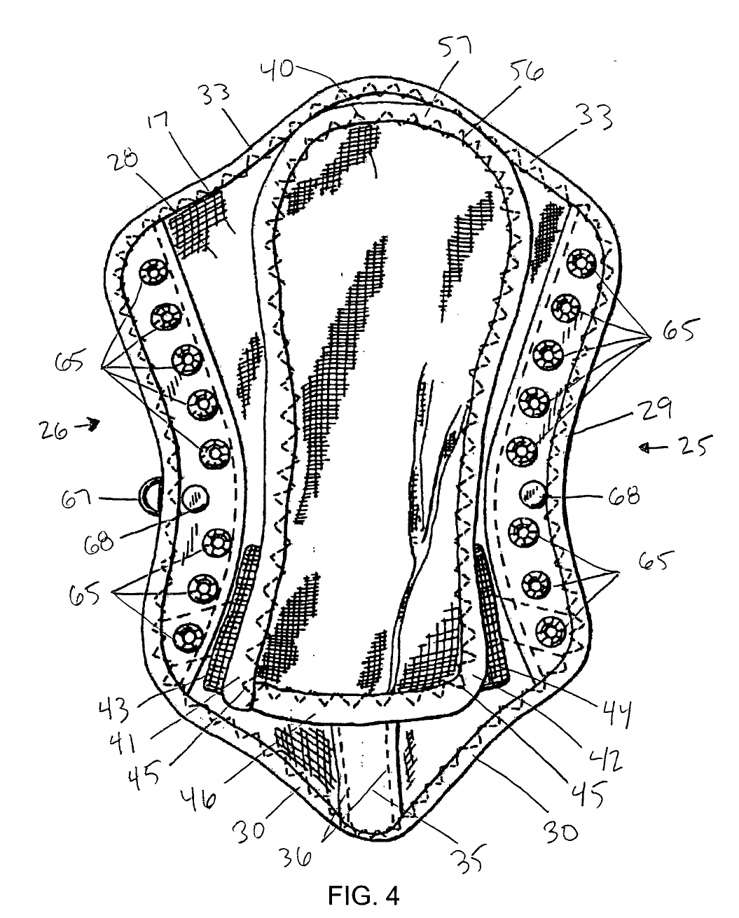

FIG. 2 shows a front view of the ankle brace 10. The ankle brace has a first side 25 and a second side 26, each with an exterior surface 27 which is preferably made of flexible but inelastic sheet material, and a lining 28 (best shown in FIG. 4) also formed of sheet material. The ankle brace 10 has a plurality of eyelet pairs 65 and a pair of loops 67 approximately in line with the eyelets and located vertically near the talus (anklebone). A shoelace 66 is threaded through the pairs of eyelets 65, tightened, and tied in a knot 69 in order to apply tension to the ankle brace to closely conform it to the shape of the foot and ankle. V-shaped relief openings 80 and curved elongate relief openings 83 are preferably formed in each side of the ankle brace. A tongue 40 is located in the front of the ankle brace. The edges of the tongue are covered with edge binding 56, preferably using stitching 57.

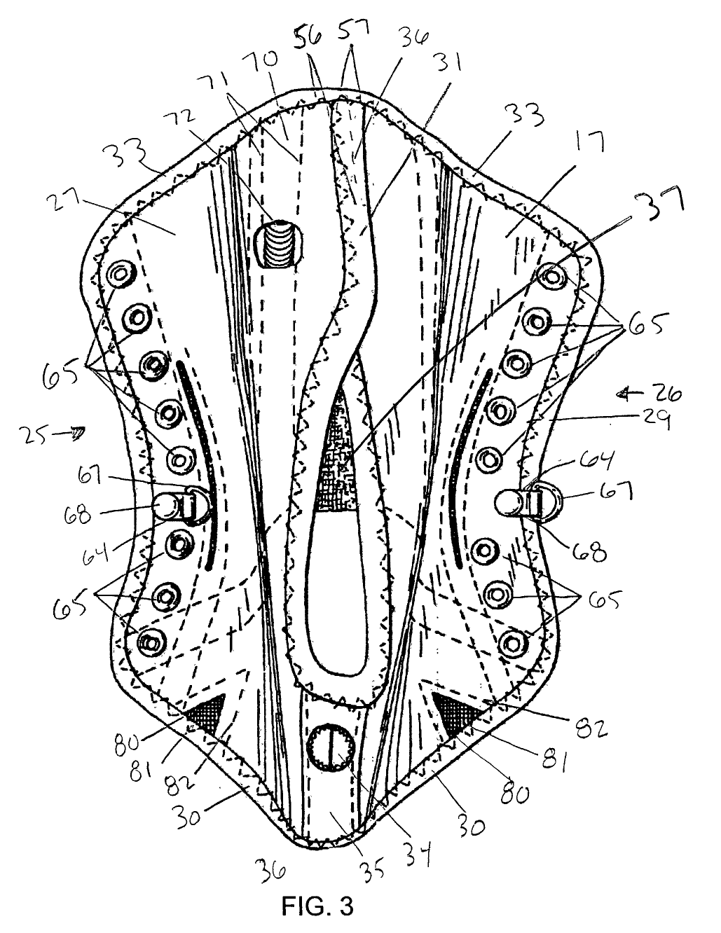

FIG. 3 shows a back view of the ankle brace pressed flat. As shown in FIG. 3, the first side 25 and the second side 26 of the base 17 are preferably generally symmetrical. Each side of the base has an upper forward edge 29, a lower forward edge 30, an upper rear edge 31 (the upper rear edge 31 of the second side 26 of the base 17 is not shown in FIG. 3), a lower rear edge 32, a top edge 33, and a bottom edge 34 (shown in cutaway in FIG. 3). The upper rear edge 31 of the first side of the base is joined to the upper rear edge 31 of the second side of the base, preferably using stitching 36. A rear elastic strip 37 is attached to the rear of the base. Although the first side 25 and second side 26 of the base 17 may be made as a single piece, they are preferably made as two separate pieces, with a bottom edge attachment member 35 that is secured, preferably using stitching 36, to join the bottom edges 34 of the first and second sides of the base to each other so that the base wraps around the foot and ankle.

As best shown in FIG. 3, the rear edges 31 of the first and second sides 25 and 26 of the base 17 of the ankle brace are fastened together, preferably using stitching 36. An edge binding 56 is secured, preferably using stitching 57, to cover the edges of the sides of the base, except for the bottom edges of the sides of the base 34 that are secured together with the bottom edge attachment member 35. The lower rear edges 32 of the first side 25 and the second side 26 of the base form an opening for the heel.

FIG. 3 also shows the loops 67 located near the forward edge 29 of the base. Each loop 67 is preferably made of metal and shaped like the letter D. Each loop 67 is preferably attached to one end of an elongate member 64. The other end of the elongate member 64 is preferably attached to the base 17 of the ankle brace using a rivet 68 which allows the loop 67 and the elongate member 64 to swivel or rotate about the rivet 68.

FIG. 3 also shows that an elongate pocket 70 is formed on each side of the base, preferably using stitching 71. As shown in the cutaway portion of FIG. 3, a resilient stay member 72 is located within the elongate pocket 70. A line of eyelets 65 appears on each side of the brace. The loops 67 and elongate members 64 may swivel or rotate about the rivet 68. V-shaped relief openings 80 are located on each side of the base. Elastic material 81 spans the V-shaped relief openings 80, and the elastic material 81 is secured to the boundaries of the relief openings 80, preferably using stitching 82.

FIG. 4 shows a front view of the ankle brace pressed flat. As best shown in FIG. 4, the ankle brace preferably has a tongue 40, of a shape which is conventional in the art, with a first lower side edge 41 and a second lower side edge 42. An edge binding 56 is secured, preferably using stitching, to cover the edges of the sides of the tongue 40. The tongue 40 is loosely attached to the base 17 by first and second stretchable elastic members 43 and 44 which are secured to the first and second lower side edges 41 and 42 of the tongue and to the first and second sides 25 and 26 of the base 17, preferably using stitching 45. The tongue 40 has a bottom edge 46. The edges of the tongue 40 are covered with edge binding 56 preferably using stitching 57. The lower forward edges 30 of the first side 25 and the second side 26 of the base (not shown in FIG. 4) and the bottom edge 46 of the tongue 40 form an opening for the toes.

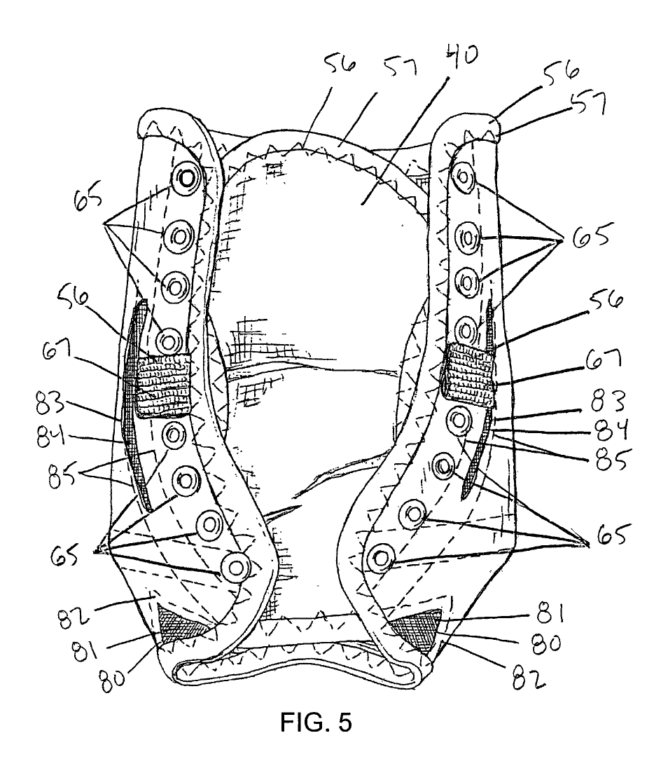

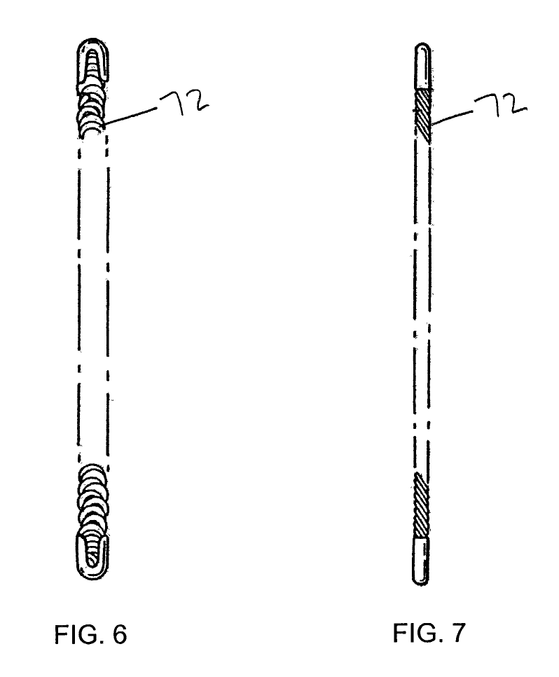

FIG. 5 shows an alternative embodiment which uses a fabric loop 67 which is attached to the base 17, preferably using stitching 57. Except for the difference in the construction of the loop 67, this alternate embodiment is otherwise the same as the preferred embodiment in FIGS. 1-4.

FIG. 8 shows the ankle brace worn under normal “high tops” footwear shown generally at 18. The footwear 18 includes footwear eyelets 20 and footwear laces 19, which are used to secure the footwear around the foot and ankle.

FIG. 9 shows the ankle brace worn under normal “high tops” footwear with the region around the loops 67 cut away to show how the laces of the footwear 19 are trained through the loops 67 to form the attachment between the ankle brace and the footwear.

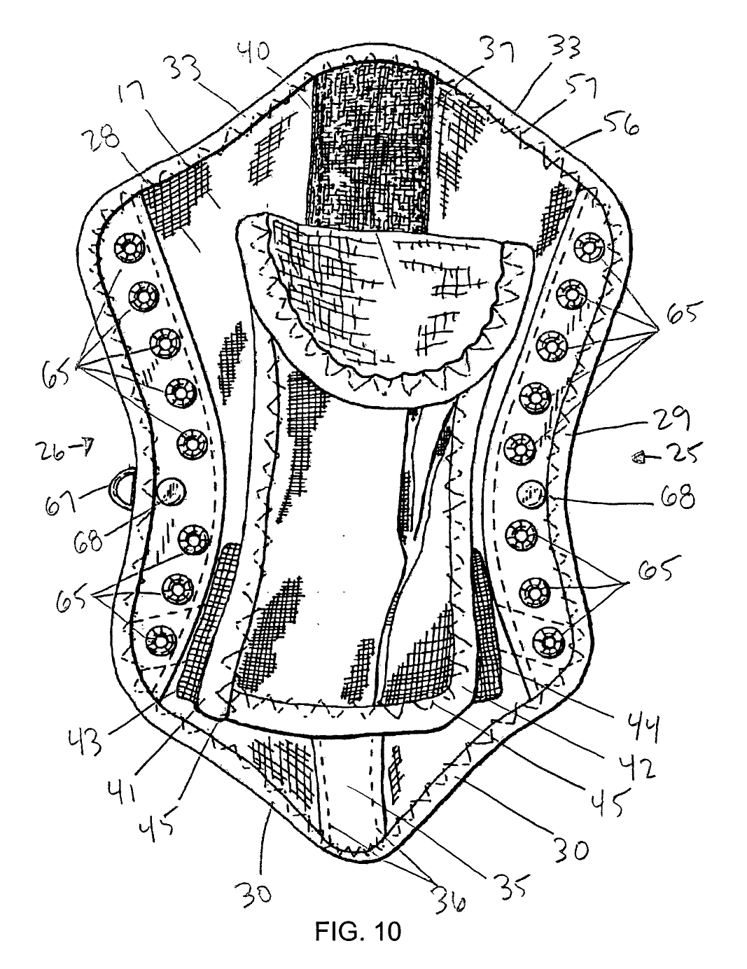

FIG. 10 shows a front view of the ankle brace pressed flat, with the tongue 40 folded down to reveal the rear elastic strip 37.

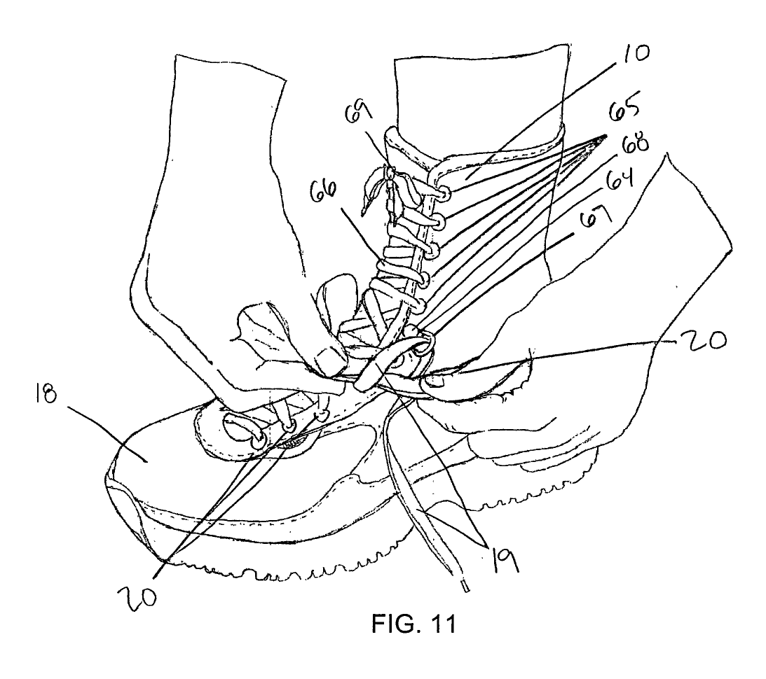

FIG. 11 shows the ankle brace worn under normal “low tops” footwear, showing how the laces of the footwear 19 are trained through the loops 67 to form the attachment between the ankle brace and the footwear.

It is understood that the invention is not confined to the embodiments set forth herein as illustrative, but embraces all such forms thereof as come within the scope of the following claims.

What is claimed is:

1. An ankle brace made to be worn inside footwear having shoelaces, comprising:
(a) a base of flexible material shaped to wrap around the rear and sides of a foot and ankle, the base having a first side and a second side, with each side of the base having a forward edge region; and
(b) at least one loop located in the vicinity of at least one of the forward edge region of the first side of the base and the forward edge region of the second side of the base such that the shoelaces of the footwear may be passed through the at least one loop.

2. The ankle brace of claim 1 wherein the loop is releasably attached to the base.

3. The ankle brace of claim 1 further comprising at least one elongate pocket on at least one of the first side of the base and the second side of the base, and a resilient stay member located in the at least one elongate pocket.

4. The ankle brace of claim 1 further comprising an elongate member having a first end and a second end, wherein the loop is attached to the first end of the elongate member and wherein the second end of the elongate member is attached to the base.

5. The ankle brace of claim 1 wherein the flexible material forming the base comprises an outer layer comprised of flexible but substantially inelastic material and an inner layer comprised of elastic material.

6. The ankle brace of claim 1 wherein the loop is D-shaped.

7. The ankle brace of claim 4 wherein the second end of the elongate member is attached to the base by a rivet.

8. The ankle brace of claim 4 wherein the elongate member swivels.

9. The ankle brace of claim 5 further comprising at least one relief opening in the outer layer of the base, each such relief opening comprised as a cut-out section of the outer layer of the base, where the inner layer spans the cut-out section and where the inner layer of elastic material is fixed to at least a portion of the perimeter of the cut-out section.

10. The ankle brace of claim 9 wherein the at least one relief opening comprises an opening in the outer layer of the base which is located on a side of the base in the vicinity of the ankle.

11. The ankle brace of claim 9 wherein the at least one relief opening comprises an opening in the outer layer of the base which is located on a side of the base in the vicinity of the lower forward edge of the base.

12. Ankle supported footwear comprising:
(a) footwear having shoelaces;
(b) a base of flexible material shaped to wrap around the rear and sides of a foot and ankle, the base having a first side and a second side, with each side of the base having a forward edge region;
(c) a means for fastening the base around the foot and ankle of the wearer; and
(d) a first loop located in the vicinity of the forward edge region of the first side of the base and a second loop located in the vicinity of the forward edge region of the second side of the base such that the shoelaces of the footwear may be passed through the first loop and the second loop.

13. The ankle brace of claim 12 comprising a first elongate pocket on the first side of the base, a first resilient stay member located in the first elongate pocket, a second elongate pocket on the second side of the base, and a second resilient stay member located in the second elongate pocket.

14. The ankle brace of claim 12 further comprising an elongate member having a first end and a second end, wherein at least one of the first loop and the second loop is attached to the first end of the elongate member and wherein the second end of the elongate member is attached to the base.

15. The ankle brace of claim 12 wherein the flexible material forming the base comprises an outer layer comprised of flexible but substantially inelastic material and an inner layer comprised of elastic material.

16. The ankle brace of claim 12 wherein at least one of the first loop and the second loop is D-shaped.

17. The ankle brace of claim 12 wherein at least one of the first loop and the second loop is releasably attached to the base.

18. The ankle brace of claim 12 wherein the means for fastening the base around the foot and ankle of the wearer comprises eyelets and laces.

19. The ankle brace of claim 14 wherein the second end of the elongate member is attached to the base by a rivet.

20. The ankle brace of claim 14 wherein the elongate member swivels.

21. The ankle brace of claim 15 further comprising at least one relief opening in the outer layer of the base, each such relief opening comprised as a cut-out section of the outer layer of the base, where the inner layer spans the cut-out section and where the inner layer of elastic material is fixed to at least a portion of the perimeter of the cut-out section.

22. The ankle brace of claim 21 wherein the at least one relief opening comprises an opening in the outer layer of the base which is located in the vicinity of the ankle.

23. The ankle brace of claim 21 wherein the at least one relief opening comprises an opening in the outer layer of the base which is located in the vicinity of the lower forward edge of the base.

24. An ankle brace made to be worn inside footwear having shoelaces, comprising:
(a) a base of flexible material shaped to wrap around the rear and sides of a foot and ankle, the base having a first side and a second side, with each side of the base having a forward edge region;
(b) a first plurality of eyelets located along the forward edge of the first side of the base, a second plurality of eyelets located along the forward edge of the second side of the base, and a shoelace such that the shoelace can be passed through the first plurality of eyelets and the second plurality of eyelets to fasten the base around the foot and ankle of the wearer; and
(c) a first loop located in the vicinity of the forward edge region of the first side of the base and a second loop located in the vicinity of the forward edge region of the second side of the base such that the shoelaces of the footwear may be passed through the first loop and the second loop.

25. The ankle brace of claim 24 comprising at least one elongate pocket on at least one of the first side of the base and the second side of the base, and at least one resilient stay member located in the at least one elongate pocket.

26. The ankle brace of claim 24 further comprising an elongate member having a first end and a second end, wherein at least one of the first loop and the second loop is attached to the first end of the elongate member and wherein the second end of the elongate member is attached to the base.

27. The ankle brace of claim 24 wherein at least one of the first loop and the second loop is D-shaped.

28. The ankle brace of claim 24 wherein at least one of the first loop and the second loop is releasably attached to the base.

29. The ankle brace of claim 24 wherein at least one of the first loop and the second loop swivels.

30. The ankle brace of claim 24 wherein the flexible material forming the base comprises an outer layer comprised of flexible but substantially inelastic material and an inner layer comprised of elastic material.

31. The ankle brace of claim 26 wherein the second end of the elongate member is attached to the base by a rivet.

32. The ankle brace of claim 26 wherein the elongate member swivels.

33. The ankle brace of claim 30 further comprising at least one relief opening in the outer layer of the base, each such relief opening comprised as a cut-out section of the outer layer of the base, where the inner layer spans the cut-out section and where the inner layer of elastic material is fixed to at least a portion of the perimeter of the cut-out section.

34. The ankle brace of claim 33 wherein the at least one relief opening comprises an opening in the outer layer of the base which is located in the vicinity of the ankle.

35. The ankle brace of claim 33 wherein the at least one relief opening comprises an opening in the outer layer of the base which is located in the vicinity of the lower forward edge of the base.

36. Ankle supported footwear comprising:
(a) footwear having shoelaces;
(b) a base of flexible material shaped to wrap around the rear and sides of a foot and ankle, the base having a first side and a second side, with each side of the base having a forward edge region;
(c) a means for fastening the base around the foot and ankle of the wearer located on the base; and
(d) a means for fastening the base to the footwear.

37. The ankle brace of claim 36 comprising at least one elongate pocket on at least one of the first side of the base and the second side of the base, and at least one resilient stay member located in the at least one elongate pocket.

38. The ankle brace of claim 36 wherein the means for fastening the base to the footwear comprises at least one loop.

39. The ankle brace of claim 36 wherein the flexible material forming the base comprises an outer layer comprised of flexible but substantially inelastic material and an inner layer comprised of elastic material.

40. The ankle brace of claim 36 further comprising an elongate member having a first end and a second end, wherein at least one of the at least one loop is attached to the first end of the elongate member and wherein the second end of the elongate member is attached to the base.

41. The ankle brace of claim 38 wherein at least one of the at least one loop swivels.

42. The ankle brace of claim 38 wherein at least one of the at least one loop is D-shaped.

43. The ankle brace of claim 38 wherein at least one of the at least one loop is releasably attached to the base.

44. The ankle brace of claim 39 further comprising at least one relief opening in the outer layer of the base, each such relief opening comprised as a cut-out section of the outer layer of the base, where the inner layer spans the cut-out section and where the inner layer of elastic material is fixed to at least a portion of the perimeter of the cut-out section.

45. The ankle brace of claim 40 wherein the second end of the elongate member is attached to the base by a rivet.

46. The ankle brace of claim 40 wherein the elongate member swivels.

47. The ankle brace of claim 44 wherein the at least one relief opening comprises an opening in the outer layer of the base which is located in the vicinity of the ankle.

48. The ankle brace of claim 44 wherein the at least one relief opening comprises an opening in the outer layer of the base which is located in the vicinity of the lower forward edge of the base.
